(19) United States
(12) Patent Application Publication
(10) Pub. No.: US 2015/0174317 A1
(43) Pub. Date: Jun. 25, 2015

(54) LIQUID TRANSPORT APPARATUS

(71) Applicant: SEIKO EPSON CORPORATION, Tokyo (JP)

(72) Inventor: Yoshihiko MOMOSE, Shiojiri-shi (JP)

(21) Appl. No.: 14/574,692

(22) Filed: Dec. 18, 2014

(30) Foreign Application Priority Data
Dec. 19, 2013 (JP) 2013-262143
Dec. 19, 2013 (JP) 2013-262144
Dec. 19, 2013 (JP) 2013-262145

(51) Int. Cl. A61M 5/142; F04B 43/12

(52) U.S. Cl. CPC A61M 5/14248; F04B 43/12; A61M 5/14228; A61M 2005/14252

(57) ABSTRACT

A liquid transport apparatus includes: a storage portion configured to store liquid; and an outflow portion arranged on an inner wall of the storage portion, the outflow portion includes a depression arranged on the inner wall of the storage portion and an opening arranged on the bottom portion of the depression. The liquid transport apparatus as such is capable of filling a storage portion with liquid efficiently.

### BACKGROUND

1. Technical Field

The present invention relates to a liquid transport apparatus.

2. Related Art

A small-sized pump for infusing drug solution such as insulin into a body is developed. Such a pump stores liquid such as insulin in a storage portion. When infusing the liquid into the storage portion, liquid is flowed in via an opening provided in the storage portion.

JP-A-2011-220140 discloses a configuration in which an opening is provided on an inner wall of the storage portion.

JP-A-2013-70714 discloses a flow channel having a constant inner diameter.

JP-A-2013-70714 discloses a configuration in which a tape surface of an adhesive portion to be adhered to the skin of a user and an outer surface of an infusing portion for infusing the drug solution to a drug solution bag are the identical line.

When filling the storage portion with liquid, how efficiently the liquid can be flowed into the storage portion against an internal pressure thereof is an issue. In other words, a liquid transport apparatus capable of allowing the liquid to flow into the storage portion efficiently against the internal pressure thereof is desired.

### SUMMARY

An advantage of some aspect of the invention is to provide a liquid transport apparatus capable of allowing liquid to flow into the storage portion efficiently against the internal pressure thereof.

An aspect of the invention is directed to a liquid transport apparatus including: a storage portion configured to store liquid therein; and an outflow portion arranged on an inner wall of the storage portion, and the outflow portion includes: a depression arranged in the inner wall, and an opening arranged on a bottom portion of the depression.

Another aspect of the invention is directed to a liquid transport apparatus including: an inflow portion configured to allow liquid to flow inward; an outflow portion provided in the storage portion configured to store the liquid; and a flow channel configured to connect the inflow portion and the outflow portion, wherein a flow rate of the liquid is set to be higher on the outflow portion side than the inflow portion side.

Other features of the invention will be apparent from the specification and attached drawings.

### BRIEF DESCRIPTION OF THE DRAWINGS

The invention will be described with reference to the accompanying drawing, wherein like numbers reference like elements.

FIG. 1 is a general perspective view of a liquid transport apparatus.

FIG. 2 is an exploded view of the liquid transport apparatus.

FIG. 3 is a cross-sectional view of the liquid transport apparatus.

FIG. 4 is a perspective top view of an interior of the liquid transport apparatus.

FIG. 5 is a schematic explanatory drawing of a pump.

FIG. 6 is an exploded perspective view illustrating an internal configuration of a main body.

FIG. 7 is a perspective view of a back surface of the main body.

FIG. 8 is an exploded perspective view illustrating an internal configuration of a cartridge.

FIG. 9 is an exploded perspective view of a back surface of the cartridge.

FIG. 10 is a perspective view of the liquid transport apparatus when viewed from a back surface side of a patch.

FIG. 11 is a cross-sectional view illustrating a supply channel of liquid into a storage portion.

FIG. 12 is a cross-sectional view taken along a line A-A in FIG. 11.

FIG. 13A is a cross-sectional view taken along a line B-B in FIG. 11. FIG. 13B is a drawing illustrating a direction in which the liquid is flowed out from an outflow portion.

FIG. 14A is an explanatory drawing of the outflow portion in a comparative example. FIG. 14B is a drawing illustrating a direction in which the liquid is flowed out from the outflow portion in the comparative example.

FIG. 15 is an explanatory drawing illustrating a cross-sectional area of an inflow portion, a flow channel and the outflow portion.

FIG. 16 is a cross-sectional view of the liquid transport apparatus including a cartridge septum.

FIG. 17A is a cross-sectional view taken along the line B-B of a second embodiment. FIG. 17B is a drawing illustrating a direction in which the liquid is flowed out from the outflow portion in the second embodiment.

FIG. 18 is a cross-sectional view taken along the line A-A of FIG. 11 of a third embodiment.

FIG. 19 is a cross-sectional view of a liquid transport apparatus including the cartridge septum of a fourth embodiment.

FIG. 20 is a cross-sectional view of the liquid transport apparatus of a fifth embodiment.

### DESCRIPTION OF EXEMPLARY EMBODIMENTS

According to the specification and the attached drawings, at least the followings become apparent.

A liquid transport apparatus includes: a storage portion configured to store liquid; and an outflow portion arranged on an inner wall of the storage portion, wherein the outflow portion including a depression arranged in the inner wall, and an opening arranged on a bottom portion of the depression.

In this manner, with the configuration in which the depression is provided on the inner wall of the storage portion and the opening is arranged on the bottom portion of the depression, liquid flowing into the storage portion from the opening can be dispersed in the depression. In this manner, by the dispersion of the liquid in the depression, a force of the liquid flowing in may be increased, so that the liquid can be flowed into the storage portion efficiently against an internal pressure in the interior thereof.

Preferably, the inner wall of the storage portion has an inclined surface shape, and the depression includes a slit-shaped portion formed to be an opening at the same height on the inclined surface.

In this configuration, since a flow channel of the liquid has the same height at the slit-shaped portion of the depression with respect to the inclined surface, the liquid can be flowed into the storage portion efficiently. In addition, since the slit-shaped portion is formed on the depression so as to have the same height as the inclined surface, the liquid can be flowed inward from the entire part of the slit-shaped portion so as to push up a film.

Preferably, the liquid is supplied from the opening to the depression and flows into the storage portion.

In this configuration, a pressure in the opening can be dispersed in the depression, and hence the liquid can be flowed into the storage portion efficiently.

Preferably, the opening is arranged at a center of the depression.

In this configuration, the liquid can be dispersed efficiently in the depression.

Preferably, the storage portion includes a curved shaped portion and the film arranged so as to extend along the curved shaped portion, and the liquid is stored between the curved shaped portion and the film.

In this configuration, the storage portion is defined between the curved shaped portion and the film. In this configuration, when the liquid is flowed between the curved shaped portion and the film, a drag is applied by the film. In this case as well, however, with the configuration described above, the liquid can be flowed into the storage portion efficiently.

Preferably, a pump configured to transport the liquid to an infused object is provided, and a supply channel for supplying the liquid to the pump is provided in the storage portion.

In this configuration, the liquid stored in the storage portion can be supplied to the pump.

Preferably, a base on which the storage portion is arranged and a flow channel connected to the opening are provided, and the flow channel is arranged on the base.

Since the flow channel is arranged on the base on which the storage portion is arranged, the storage portion and the flow channel can be coupled with a same member.

Preferably, a lid member arranged in the flow channel is provided.

In this configuration, in the case where the liquid is flowed into the storage portion via the flow channel, the liquid can be prevented from leaking from the flow channel.

A liquid transport apparatus includes: an inflow portion configured to allow liquid to flow inward; an outflow portion provided in the storage portion configured to store the liquid; and a flow channel configured to connect the inflow portion and the outflow portion, wherein a flow rate of the liquid is set to be higher on the outflow portion side than the inflow portion side.

In this configuration, since the flow rate on the outflow portion side provided in the storage portion is set to be higher than that on the inflow portion side, the liquid can be flowed into the storage portion efficiently against the internal pressure therein.

Preferably, a lid member is arranged at the inflow portion.

In this configuration, the liquid is prevented from leaking from the inflow portion side after the liquid has been infused from the inflow portion.

Preferably, the lid member is a septum.

In this configuration, the liquid can be infused with a needle-like tool via the septum.

Preferably, the cross-sectional area on the outflow portion side may be set to be smaller than that on the inflow portion side.

In this configuration, the flow rate on the outflow portion side may be set to be higher than the flow rate on the inflow portion side.

Preferably, the storage portion includes a curved shaped portion and a film arranged so as to extend along the curved shaped portion, and the liquid is stored between the curved shaped portion and the film.

In this configuration, the storage portion is defined between the curved shaped portion and the film. In this configuration, when the liquid is flowed between the curved shaped portion and the film, a drag is applied by the film. In this case as well, however, with the configuration described above, the liquid can be flowed into the storage portion efficiently.

Preferably, the outflow portion includes a depression arranged on an inner wall of the storage portion and an opening arranged on a bottom portion of the depression.

In this configuration, the liquid flowing into the storage portion from the opening may be dispersed in the depression. In this manner, by the dispersion of the liquid in the depression, a force of the liquid flowing in is increased, so that the liquid can be flowed into the storage portion efficiently against an internal pressure in the interior thereof.

Preferably, the inner wall of the storage portion has an inclined surface shape, and the depression includes a slit-shaped portion formed to be an opening at the same height on the inclined surface.

In this configuration, since a flow channel of the liquid has the same height at the slit-shaped portion of the depression with respect to the inclined surface, the liquid can be flowed into the storage portion efficiently. In addition, since the slit-shaped portion is formed on the depression so as to have the same height as the inclined surface, the liquid can be flowed inward from the entire part of the slit-shaped portion so as to push up a film.

Preferably, a base on which the storage portion is arranged is provided and the flow channel is arranged on the base.

In this configuration, since the flow channel is arranged on the base on which the storage portion is arranged, the storage portion and the flow channel can be coupled with a same member.

A liquid transport apparatus capable of being mounted on a biological body, and configured to transport liquid to the biological body, includes: a storage portion configured to store the liquid therein; and a lid member configured to close an opening which opens toward the biological body and configured to allow a needle for infusing the liquid into the storage portion to pick therethrough, wherein a gap is provided between an outer surface of the lid member on the biological body side and a mounting surface of the liquid transport apparatus with respect to the biological body.

With the liquid transport apparatus configured as described above, since the outer surface of the lid member is away from a biological body surface by a size corresponding to the size of the gap, the lid member does not come into contact with the biological body surface, and hence the probability of contamination of the lid member with bacteria or the like is reduced. Since the outer surface of the lid member does not come into contact with outside air, a probability that the lid member is deteriorated by the outside air is reduced.

Preferably, a peripheral surface of the opening is chamfered.

With the liquid transport apparatus configured as described above, a space which constitutes the gap between the biological body surface and the lid member is increased by a volume corresponding to the chamfered portion, the probability of contamination of the lid member with bacteria or the like is further reduced. Since the opening is gradually narrowed and the inner side is closed by the lid member, the needle for infusing the liquid into the storage portion can easily pick the lid member.

Preferably, the lid member is interposed between the two members overlapped in the direction of picking of the needle.

With the liquid transport apparatus configured as described above, the lid member is interposed and fixed between the two members, and hence one of the members (the member on the storage portion side) supports the lid member when picking the needle and, on the contrary, the other member (the member on the biological body side) supports the lid member when pulling out the needle, so that the stable fixation of the lid member is achieved.

Preferably, the opening is covered with the adhesive sheet configured to attach the liquid transport apparatus to the biological body.

With the liquid transport apparatus configured as described above, since the outer surface of the lid member is isolated from the outside by the adhesive sheet, a probability of contamination of the lid member with bacteria or the like or deterioration of the lid member due to the outside air is eliminated.

### FIRST EMBODIMENT

### Basic Configuration of Liquid Transport Apparatus

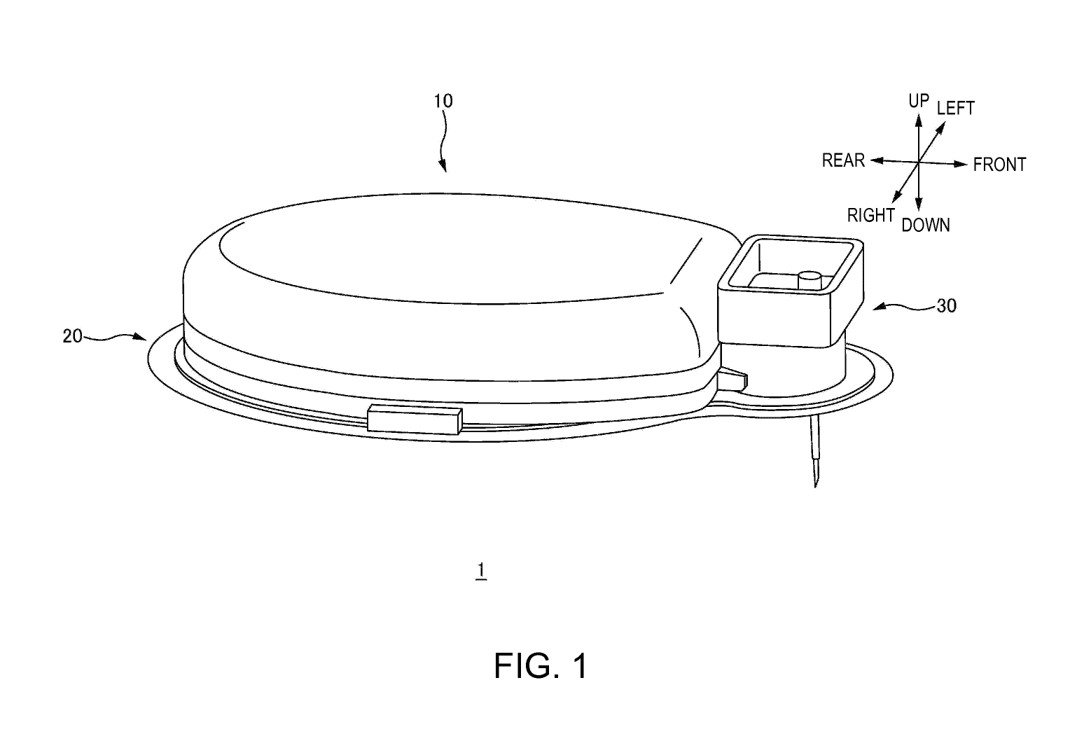

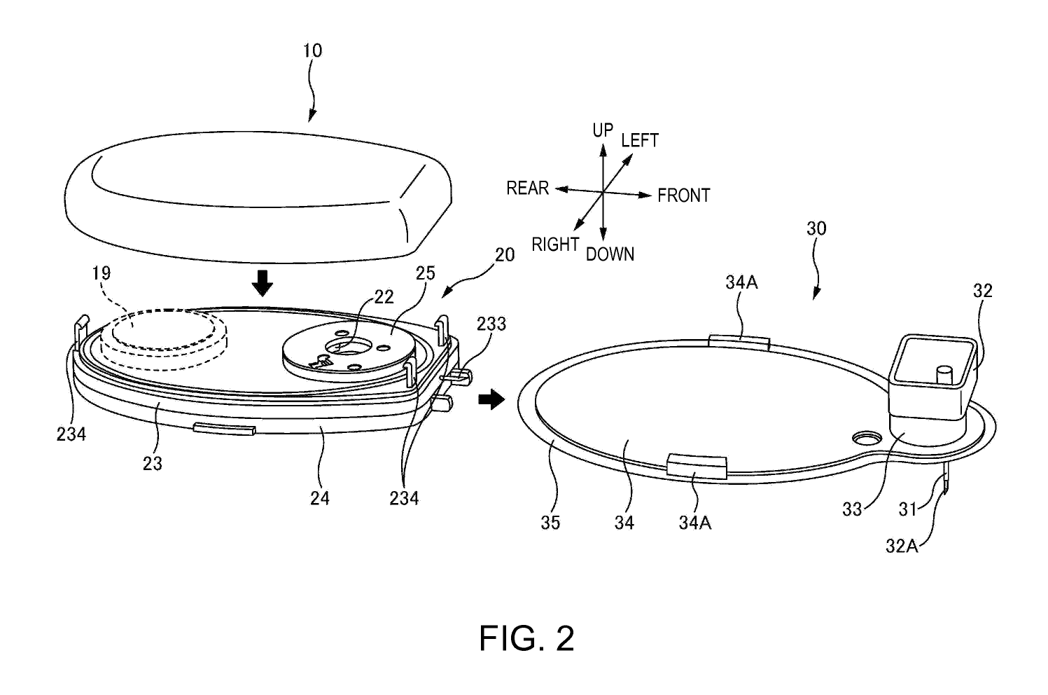

FIG. 1 is a general perspective view of a liquid transport apparatus 1. FIG. 2 is an exploded view of the liquid transport apparatus 1. As illustrated in these drawings, a side (biological body side) where the liquid transport apparatus 1 is adhered is referred to as “down” and a opposite side may be referred to as “up” in the description. As illustrated in these drawings and drawings to be given below, description may be given by using directions of front, rear, left and right.

The liquid transport apparatus 1 is an apparatus configured to transport liquid. The liquid transport apparatus 1 includes a main body 10, a cartridge 20, and a patch 30. The main body 10, the cartridge 20, and the patch 30 are separable as illustrated in FIG. 2, and are assembled integrally when in use as illustrated in FIG. 1. The liquid transport apparatus 1 is preferably used for infusing insulin stored in the cartridge 20 regularly, for example by adhering the patch 30 to the biological body. When the liquid (for example, insulin) stored in the cartridge 20 is finished up, the cartridge 20 is replaced. The patch 30 is also replaced. In contrast, the main body 10 is used continuously.

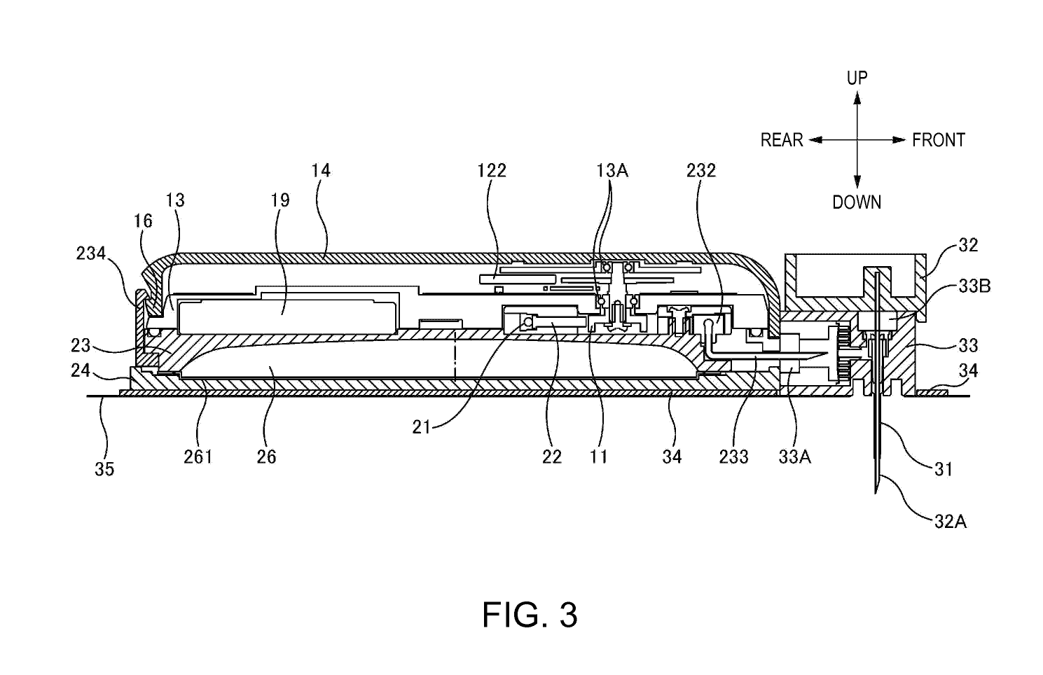

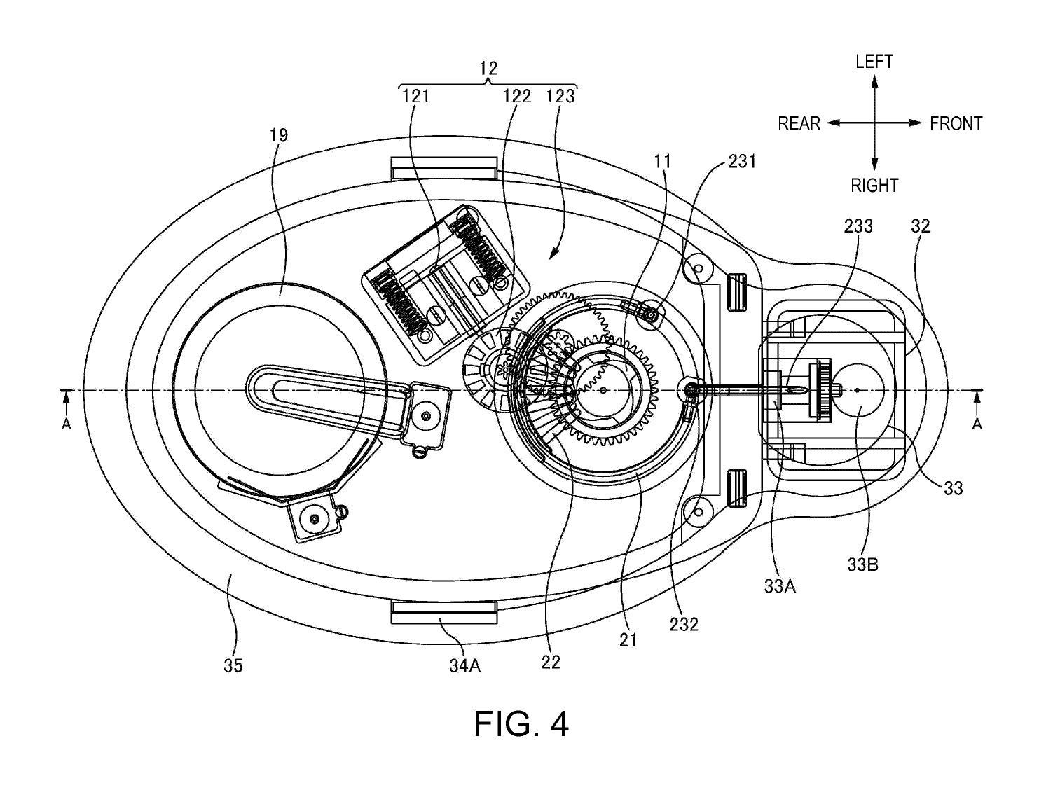

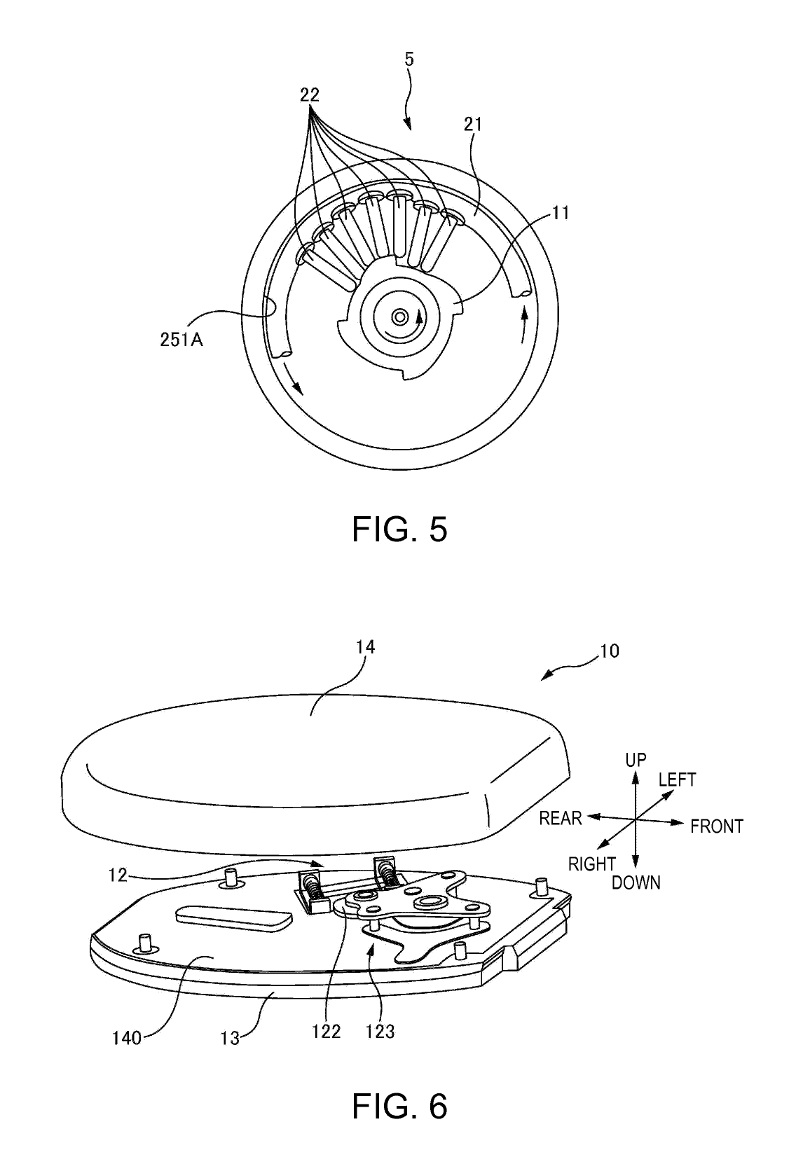

FIG. 3 is a cross-sectional view of the liquid transport apparatus 1. FIG. 4 is a perspective top view of the interior of the liquid transport apparatus 1. FIG. 4 also illustrates a configuration of a pump unit 5. FIG. 5 is a schematic explanatory drawing of the pump unit 5.

The pump unit 5 has a function as a pump for transporting liquid stored in the cartridge 20, and includes a tube 21, a plurality of fingers 22, a cam 11, and a drive mechanism 12.

The tube 21 is a tube for transporting liquid. An upstream side of the tube 21 (the upstream side with reference to a direction of transport of the liquid) communicates with a storage portion 26 of the liquid in the cartridge 20. The tube 21 has a resiliency to an extent to close when pressed by the fingers 22 and restore when the force from the fingers 22 is released. The tube 21 is arranged in a partially arcuate shape along an inner surface of a tube guide wall 251A of the cartridge 20. An arcuate portion of the tube 21 is arranged between an inner surface of the tube guide wall 251A and a plurality of fingers 22. A center of the arc of the tube 21 matches a center of rotation of the cam 11.

The fingers 22 are members for closing the tube 21. The fingers 22 are driven upon reception of a force from the cam 11. The fingers 22 each include a rod-shaped shaft portion and a flange-shaped pressing portion and are formed into a T-shape. The rod-shaped shaft portion comes into contact with the cam 11, and the flange-shaped pressing portion comes into contact with the tube 21. The fingers 22 are supported so as to be movable along an axial direction.

The plurality of fingers 22 are arranged radially from the center of rotation of the cam 11 at regular distance. The plurality of fingers 22 are arranged between the cam 11 and the tube 21. Here seven fingers 22 are provided. From the upstream side of a direction of transport of the liquid, these fingers are denoted in sequence as a first finger 22A, a second finger 22B, . . . and a seventh finger 22G.

The cam 11 has projecting portions at four positions on an outer periphery thereof. The plurality of fingers 22 are arranged on the outer periphery of the cam 11, and the tube 21 is arranged on the outside of the fingers 22. The fingers 22 are pressed by the projecting portions of the cam 11, so that the tube 21 is closed. When the fingers 22 come out of contact with the projecting portions, the tube 21 is restored to the original shape by a resiliency of the tube 21. When the cam 11 rotates, the seven fingers 22 are pressed in sequence by the projecting portions, and close the tube 21 in sequence from the upstream side in the direction of transport. Accordingly, when the tube 21 is caused to perform a peristaltic action, and liquid is compressed and transported to the tube 21. In order to prevent a reverse flow of the liquid, projecting portions of the cam 11 are formed so that at least one, preferably two fingers 22 close the tube 21.

A drive mechanism 12 is a mechanism for driving the cam 11 to rotate. The drive mechanism 12 includes a piezoelectric motor 121, a rotor 122, and a deceleration transmitting mechanism 123 (see FIG. 4).

The piezoelectric motor 121 is a motor for rotating the rotor 122 by using vibrations of a piezoelectric element. The piezoelectric motor 121 vibrates a vibrator by applying a drive signal on the piezoelectric elements adhered to both surfaces of the rectangular vibrator. An end portion of the vibrator comes into contact with the rotor 122, and when the vibrator vibrates, the end portion vibrates while tracing out a predetermined orbit such as an oval orbit or a figure eight orbit. By the end portion of the vibrator coming into contact with the rotor 122 at a portion of the vibration orbit, the rotor 122 is driven to rotate. The piezoelectric motor 121 is biased toward the rotor 122 by a pair of springs so that the end portion of the vibrator comes into contact with the rotor 122.

The rotor 122 is a driven member rotated by the piezoelectric motor 121. The rotor 122 is provided with a rotor pinion which constitutes part of the deceleration transmitting mechanism 123.

The deceleration transmitting mechanism 123 is a mechanism configured to transmits a rotation of the rotor 122 to the cam 11 at a predetermined reduction ratio. The deceleration transmitting mechanism 123 includes the rotor pinion, a transmitting wheel, and a cam gear. The rotor pinion is a small gear integrally mounted on the rotor 122. The transmitting wheel includes a large gear that engages the rotor pinion and a pinion that engages the cam gear, and has a function to transmit rotational force of the rotor 122 to the cam 11. The cam gear is integrally mounted on the cam 11, and is rotatably supported together with the cam 11.

The pump unit 5 includes the tube 21, the plurality of fingers 22, the cam 11 and the drive mechanism 12, and the cam 11 and the drive mechanism 12 are provided on the main body 10, and the tube 21 and the plurality of fingers 22 are provided on the cartridge 20. Configurations of the main body 10, the cartridge 20, and the patch 30 will be described.

### Main Body 10

FIG. 6 is an exploded perspective view illustrating an internal configuration of a main body 10. FIG. 7 is a perspective view of a back surface of the main body 10. Referring now to FIG. 1 to FIG. 4, the configuration of the main body 10 will be described.

The main body 10 includes a main body base 13 and a main body case 14. On the main body base 13, the above-described drive mechanism 12 and a control substrate 15 (control unit) for performing control such as the piezoelectric motor 121 are provided. The drive mechanism 12 (the piezoelectric motor 121, the rotor 122, the deceleration transmitting mechanism 123) on the main body base 13 and the control substrate 15 are protected by being covered with the main body case 14.

The main body base 13 is provided with a bearing 13A. A rotating shaft of the cam 11 penetrates through the main body base 13, and the bearing 13A supports the rotating shaft of the cam 11 so as to be rotatable with respect to the main body base 13. The cam 11 is integrally formed with the cam gear which constitutes part of the deceleration transmitting mechanism 123, and the cam gear is arranged in the interior of the main body 10 by being covered with the main body case 14, and the cam 11 is exposed from the main body 10. When the main body 10 and the cartridge 20 are combined, the cam 11 exposed from the main body 10 engages the end portions of the fingers 22 of the cartridge 20.

The main body 10 is provided with a hook catch 16. A fixing hook 234 of the cartridge 20 is caught by the hook catch 16, whereby the main body 10 is fixed to the cartridge 20.

The main body 10 includes a battery housing 18. A battery 19 housed in the battery housing 18 is a power source of the liquid transport apparatus 1.

### Cartridge 20

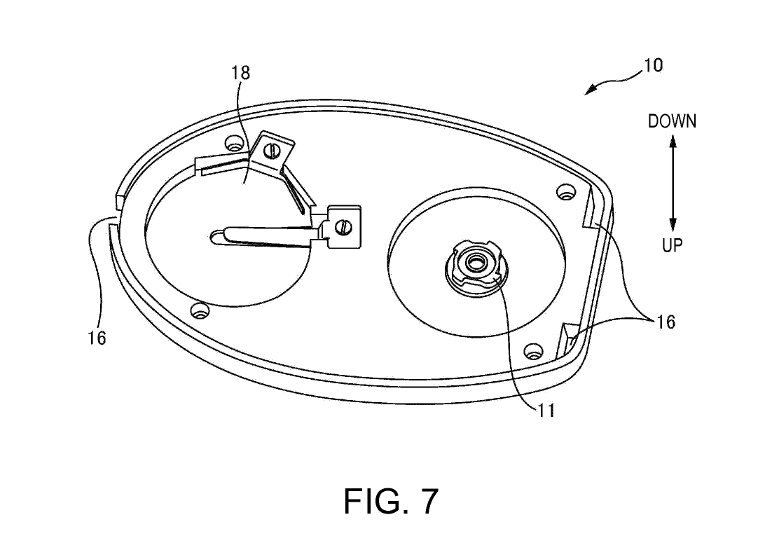

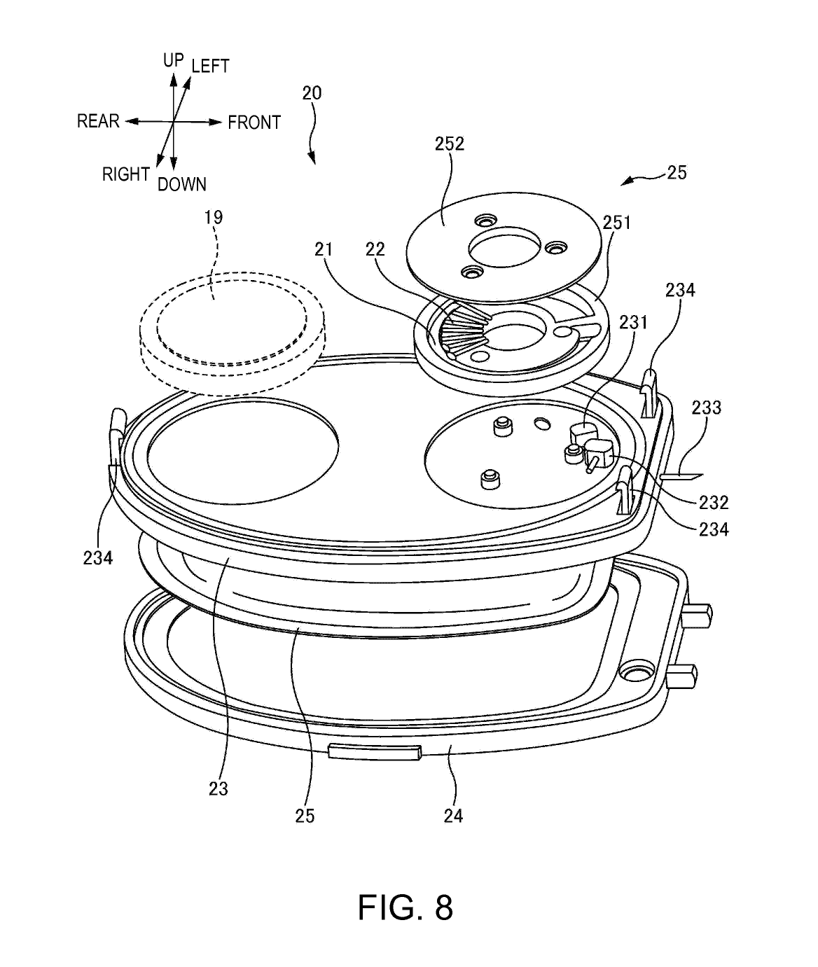

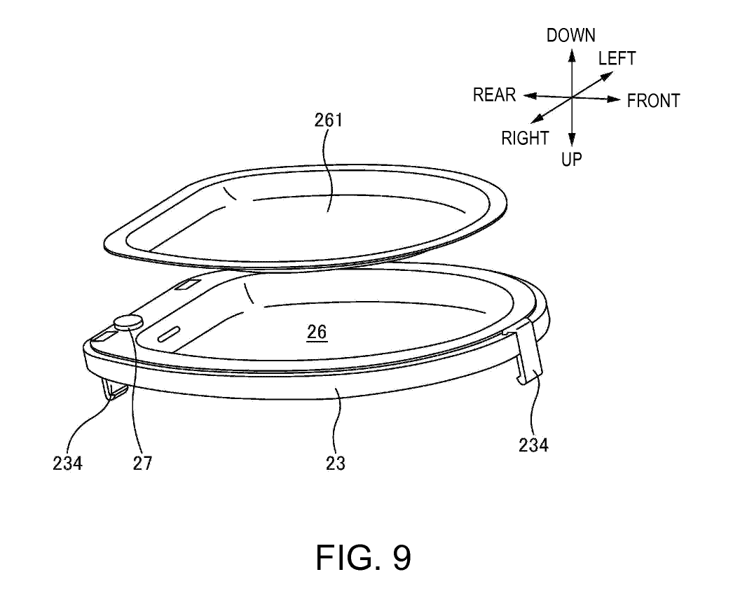

FIG. 8 is an exploded perspective view illustrating an internal configuration of the cartridge 20. FIG. 9 is an exploded perspective view of a back surface of a cartridge 20. Referring now to FIG. 1 to FIG. 5, the configuration of the cartridge 20 will be described.

The cartridge 20 includes a cartridge base 23 and a base receiver 24.

A tube unit 25 is provided on an upper side of the cartridge base 23. The tube unit 25 includes the tube 21 and the plurality of fingers 22 described above, a unit base 251, and a unit cover 252. The unit base 251 is provided with the tube guide wall 251A, and the tube 21 is arranged in an arcuate shape in the interior of the unit base 251. The unit base 251 supports the fingers 22 so as to be movable in the axial direction. The tube 21 and the fingers 22 in the unit base 251 are covered with the unit cover 252.

The tube unit 25 has a flat cylindrical shape, and the cam 11 exposed from the main body 10 is inserted into a cavity at a center of the tube unit 25. Accordingly, the cam 11 on a main body 10 side and the fingers 22 on a cartridge 20 side engage each other.

The cartridge base 23 is provided with a supply-side joint 231 and a discharge side joint 232. The ends of the tube 21 in the tube unit 25 are connected to the supply-side joint 231 and the discharge side joint 232. When the plurality of fingers 22 compress the tube 21 in sequence, liquid is supplied from the supply-side joint 231 to the tube 21, and the liquid is discharged from the discharge side joint 232. A connecting needle 233 communicates with the discharge side joint 232, and the liquid discharged from the discharge side joint 232 is supplied to the patch 30 side via the connecting needle 233.

The cartridge base 23 is provided with a fixing hook 234 formed thereon. The fixing hook 234 is caught by the hook catch 16 of the main body 10, whereby the main body 10 is fixed to the cartridge 20.

A reserve film 261 is interposed between the cartridge base 23 and the base receiver 24. The periphery of the reserve film 261 is tightly bonded to the bottom surface of the cartridge base 23. The storage portion 26 is formed between the cartridge base 23 and the reserve film 261, and the liquid (for example, insulin) is stored in the storage portion 26. The storage portion 26 communicates with the supply-side joint 231, and the liquid stored in the storage portion 26 is supplied to the tube 21 via the supply-side joint 231.

As described above, the storage portion 26 is provided on a lower side of the cartridge base 23. Since the tube 21 and the fingers 22 which constitute part of the pump unit 5 are arranged on an upper side of the cartridge base 23, the pump unit 5 and the storage portion 26 are provided above and below. Accordingly, the liquid transport apparatus 1 may be reduced in size. The storage portion 26 is arranged on the biological body side of the pump unit 5. Accordingly, the liquid stored in the storage portion 26 can easily be warmed by body temperature of the biological body, so that the difference between the temperature of the liquid and the temperature of the biological body is restrained.

When the liquid stored in the storage portion 26 is used up, the cartridge 20 is removed from the liquid transport apparatus 1, and is replaced by a new cartridge 20. However, liquid can be infused into the storage portion 26 by using an injection needle from the outside via a cartridge septum 27. The cartridge septum 27 includes a material which closes a hole when the injection needle is pulled out (for example, butyl rubber, isoprene rubber, silicone rubber, thermoplastic elastomer and the like).

### Patch 30

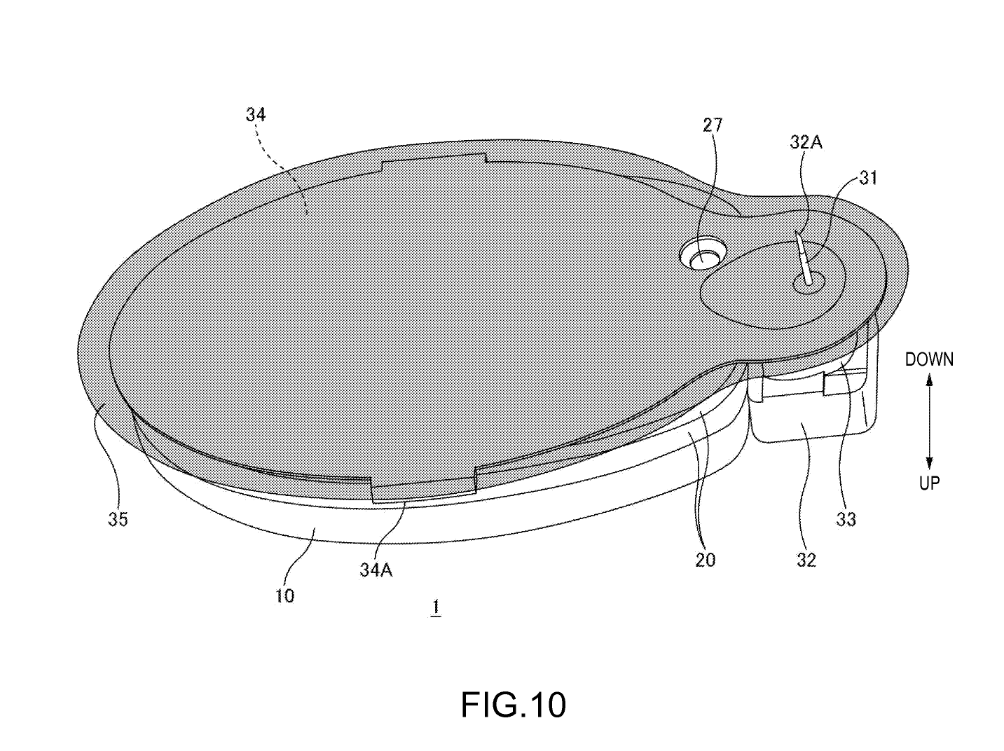

FIG. 10 is a perspective view of the liquid transport apparatus 1 when viewed from the bottom surface side of the patch 30. Referring now to FIG. 1 to FIG. 5, a configuration of the patch 30 will be described.

The patch 30 includes a catheter 31, an introduction needle folder 32, a port base 33, a patch base 34, and an adhesive pad 35.

The catheter 31 is a tube for infusing liquid into the biological body. The catheter 31 is formed of, for example, a soft material such as fluorine-contained resin or the like. One end of the catheter 31 is fixed to the port base 33.

The introduction needle folder 32 is a member configured to hold an introduction needle 32A. An end of the introduction needle 32A is fixed to the introduction needle folder 32. The introduction needle 32A is a metallic needle for inserting the soft catheter 31 into the biological body. The introduction needle 32A is an elongated hollow tube-shaped needle and has a lateral hole, which is not illustrated. When liquid is supplied from the lateral hole of the introduction needle 32A, the liquid is discharged from a distal end of the introduction needle 32A. Accordingly, a priming treatment which fills the flow channel of the liquid transport apparatus 1 with the liquid before picking the catheter 31 into the biological body may be performed.

In a state before usage, the introduction needle folder 32 is mounted on the port base 33. The introduction needle 32A is inserted into the catheter 31 and a needle point is exposed from a lower side of the catheter 31. When adhering the patch 30 to the biological body, the catheter 31 is picked into the biological body together with the introduction needle 32A, and then the introduction needle folder 32 is pulled out from the port base 33 together with the introduction needle 32A (pulled out). Since the hard introduction needle 32A does not need to dwell in the biological body, a load applied to the biological body is small. Although the catheter 31 continuously dwells in the biological body, since the catheter 31 is soft, a load applied to the biological body is small.

The port base 33 is a member configured to supply liquid supplied from the connecting needle 233 of the cartridge 20 to the catheter 31. The port base 33 includes a connecting needle septum 33A and an introduction needle septum 33B. The connecting needle septum 33A and the introduction needle septum 33B include a material which closes a hole when the injection needle is pulled out (for example, butyl rubber, isoprene rubber, silicone rubber, thermoplastic elastomer or the like). The connecting needle septum 33A includes the connecting needle 233 passed through the cartridge 20, and the liquid is supplied from the cartridge 20 side to the patch 30 side beyond the connecting needle septum 33A via the connecting needle 233. Even when the connecting needle 233 of the cartridge 20 is pulled out from the patch 30 for the replacement of the cartridge 20, the hole of the connecting needle septum 33A by the connecting needle 233 of the cartridge 20 is naturally closed. The introduction needle septum 33B includes the introduction needle 32A inserted therein. When the introduction needle 32A is pulled out, a hole formed by the introduction needle 32A in the introduction needle septum 33B is spontaneously closed. The connecting needle septum 33A and the introduction needle septum 33B prevent liquid in the patch 30 from leaking outward or body liquid of the biological body from flowing reversely toward the patch 30. An area of the port base 33 where the introduction needle 32A is present (area other than the introduction septum) serves as a flow channel of liquid after the introduction needle 32A is pulled out.

The patch base 34 is a flat panel-shaped member fixed to the port base 33. The patch base 34 includes a fixing portion 34A for fixing the base receiver 24. The adhesive pad 35 is attached to a bottom surface of the patch base 34. The adhesive pad 35 is an adhesive pad to adhere the patch 30 to the biological body or the like.

In the liquid transport apparatus 1 described above, the pump unit 5 and the storage portion 26 are arranged above and below, and hence the reduction in size of the liquid transport apparatus 1 is achieved. Accordingly, the compact adhesive pad 35 is achieved.

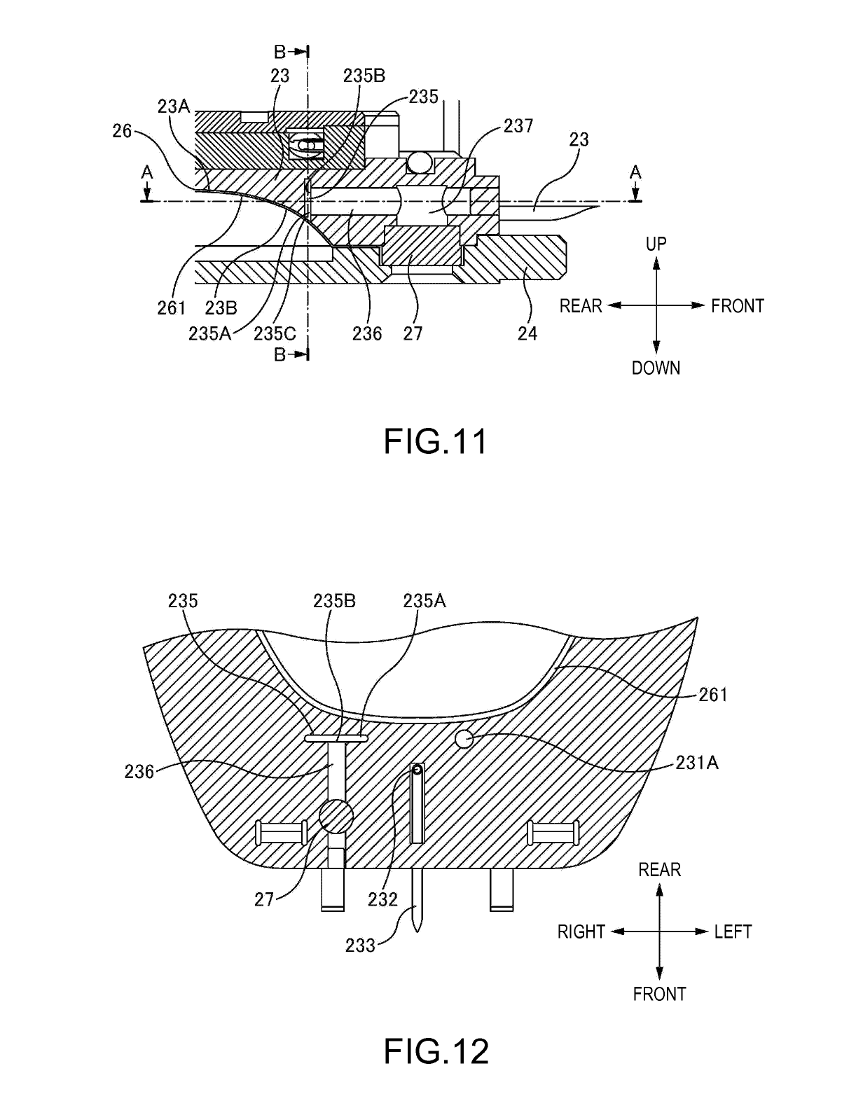

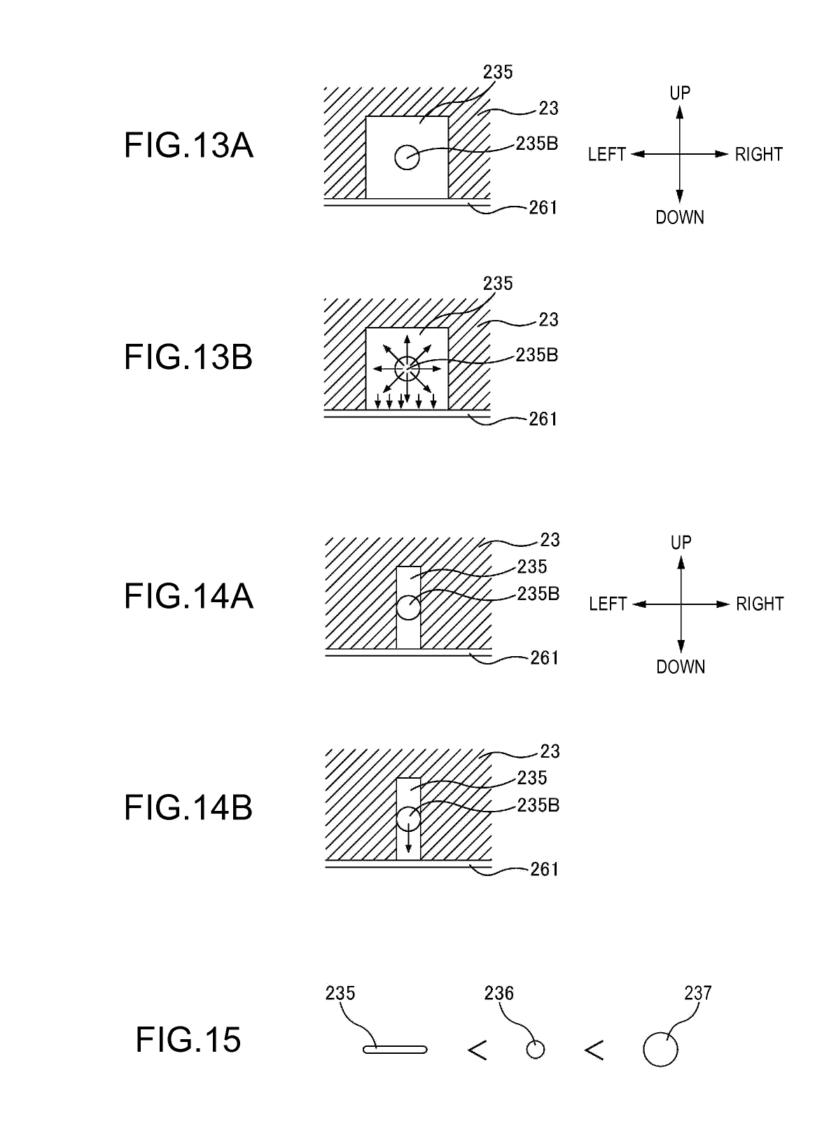

FIG. 11 is a cross-sectional view illustrating a supply channel of liquid into a storage portion 26. FIG. 12 is a cross-sectional view taken along a line A-A in FIG. 11. FIG. 13A is a cross-sectional view taken along a line B-B in FIG. 11. FIG. 13B is a drawing illustrating a direction in which the liquid is flowed out from an outflow portion 235. Referring now to these drawings, the supply channel of the liquid to the storage portion 26 will be described.

The storage portion 26 is defined by a curved shaped portion 23A provided on the cartridge base 23 (which corresponds to a base) and the reserve film 261 arranged so as to extend along the curved shaped portion 23A. When the liquid is not stored, the curved shaped portion 23A and the reserve film 261 are almost in tight contact with each other. In contrast, when the liquid flows into the storage portion 26, the reserve film 261 is separated from the curved shaped portion 23A, and the storage portion 26 between the curved shaped portion 23A and the reserve film 261 is increased.

FIG. 11 illustrates a state in which the liquid is not stored between the curved shaped portion 23A and the reserve film 261, and FIG. 3 described above illustrates a state in which the storage portion is filled with the liquid and is expanded to the maximum.

The outflow portion 235 is formed on the inner wall of the storage portion 26. The liquid flows out from the outflow portion 235 to the storage portion 26. The outflow portion 235 includes a depression 235A and an opening 235B. The depression 235A is provided on the inner wall of the storage portion 26. The opening 235B is provided at a center of a bottom portion of the depression 235A. The term “bottom portion” here has a bottom portion side on the side toward a flow channel 236, and in FIG. 11, the opening 235B is an opening formed on the front side of the depression 235A.

The curved shaped portion 23A as an inner wall of the storage portion 26 has an inclined surface 23B. The depression 235A includes a slit-shaped portion 235C formed so as to be an opening having the same height as the inclined surface 23B. In this configuration, the liquid is supplied from the opening 235B to the depression 235A, and is dispersed into the depression 235A, and then flows into the storage portion 26.

The storage portion 26 is provided with a supply channel 231A for supplying liquid to the above-described pump unit 5.

The cartridge base 23 is provided with the outflow portion 235 described above, and the flow channel 236 communicating with the outflow portion 235 and, in addition, with an inflow portion 237 communicating with the flow channel 236. An inflow portion 237 is a flow channel extending in the vertical direction. In contrast, the flow channel 236 is a flow channel extending in the fore-and-aft direction so as to connect the opening 235B and the inflow portion 237. Since the flow channel 236 is arranged on the cartridge base 23 on which the storage portion 26 is arranged in this manner, the storage portion 26 and the flow channel 236 can be formed of the same member.

The inflow portion 237 is provided with the cartridge septum 27 (which corresponds to the lid member). Accordingly, liquid can be infused into the inflow portion 237 with a syringe or the like via the cartridge septum 27. Then the infused liquid can be fed to the storage portion 26, and liquid can be prevented from leaking from the inflow portion 237.

In this configuration, the storage portion 26 can be formed between the curved shaped portion 23A and the reserve film 261. However, since the reserve film 261 is arranged so as to extend along the curved shaped portion 23A, when the liquid is flowed between the curved shaped portion 23A and the reserve film 261, a drag generated by the reserve film 261 is applied to the liquid.

However, in the first embodiment, since the depression 235A is provided on the inner wall of the storage portion 26 and the opening 235B is arranged on the bottom portion of the depression 235A, liquid flowing into the storage portion 26 from the opening 235B can be dispersed in the depression 235A. Since the flow channel of the liquid is at the same height as the inclined surface 23B at the slit-shaped portion 235C of the depression 235A, the liquid dispersed in the depression 235A flows in so as to spread to the left and the right direction in the storage portion 26. The liquid flowed therein spreads over the entire curved shaped portion 23A in the storage portion 26. In addition, when the liquid flows in, the entire liquid spread over the entire part of the curved shaped portion 23A presses the reserve film 261 upward (press downward in FIG. 11). With this configuration, the force of the liquid flowing in is increased so that the liquid can be flowed in efficiently against the inner pressure of the storage portion 26.

FIG. 14A is an explanatory drawing of the outflow portion 235 in a comparative example. FIG. 14B is a drawing illustrating a direction in which the liquid is flowed out from the outflow portion 235 in the comparative example. As illustrated in FIGS. 14A and 14B, in the case where the shape of the outflow portion 235 has substantially the same width as the opening 235B, and extends to the storage portion 26 without changing the width, the liquid proceeds toward the storage portion 26 without dispersing at the outflow portion 235. In this case, the liquid flowed into the storage portion 26 can hardly spread to the left and the right direction in the storage portion 26. Therefore, the liquid can hardly be distributed over the entire part of the curved shaped portion 23A, and hence can hardly push the reserve film 261 upward.

Subsequently, a relationship of the cross-sectional areas among the inflow portion 237, the flow channel 236 and the outflow portion 235 will be described.

FIG. 15 is an explanatory drawing of the cross-sectional areas of the inflow portion 237, the flow channel 236, and the outflow portion 235. FIG. 15 illustrates the cross-sectional shapes and the magnitude relation among the cross-sectional areas thereof. The cross-sectional shapes of the inflow portion 237 and the outflow portion 235 are the same as those illustrated in FIG. 12 described above. The cross-sectional shape of the flow channel 236 illustrated in this drawing is that of the flow channel 236 illustrated in FIG. 11 cut in the vertical direction.

The cross-sectional area of the outflow portion 235 is smaller than the cross-sectional area of the flow channel 236. The cross-sectional area of the flow channel 236 is smaller than the cross-sectional area of the inflow portion 237. Since the cross-sectional area is reduced from the inflow portion 237 toward the outflow portion 235 in this manner, when the liquid is infused into the inflow portion 237, the flow rate of the liquid is increased as it approaches the outflow portion 235.

Therefore, since the flow rate on the outflow portion 235 side is set to be faster than that of the inflow portion 237 side in the first embodiment, the reserve film 261 adhered to the curved shaped portion 23A of the cartridge base 23 can easily be separated from the curved shaped portion at a portion of high flow rate. Therefore, the liquid can be flowed in efficiently against the internal pressure of the storage portion 26.

The arrangement of the cartridge septum 27 will be described.

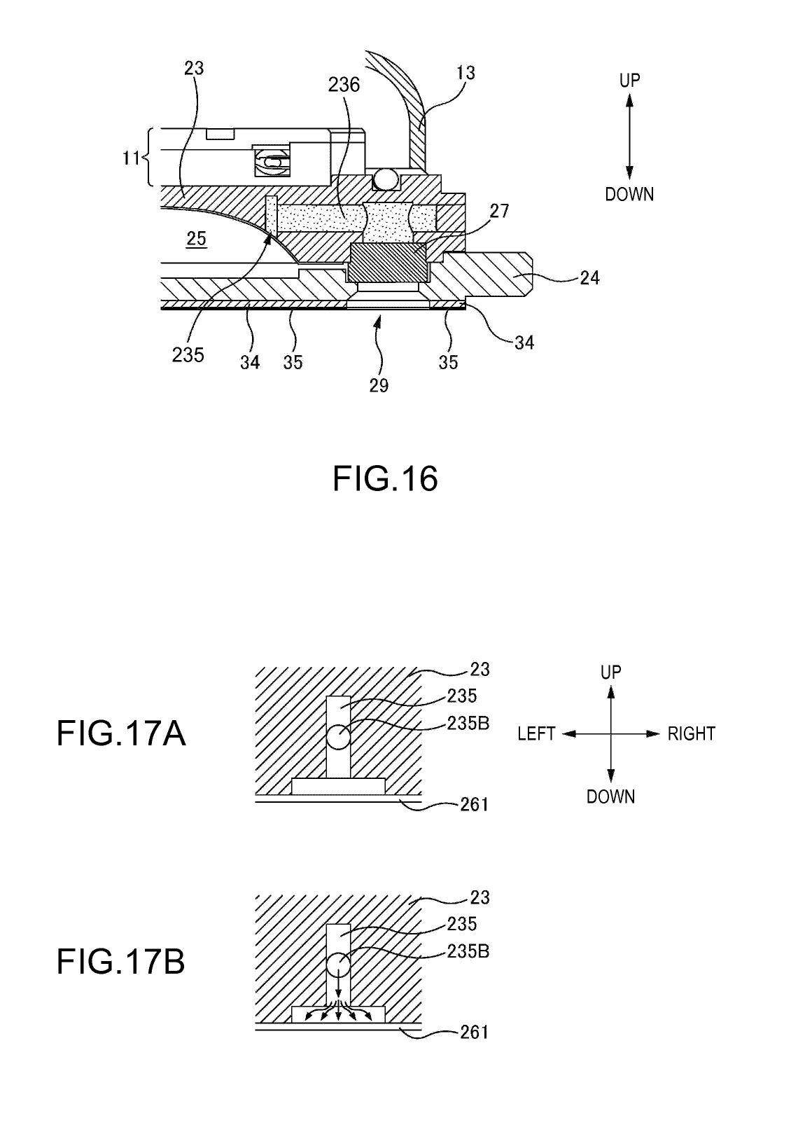

FIG. 16 is a cross-sectional view of the liquid transport apparatus 1 including the cartridge septum 27. As illustrated in FIG. 10 and FIG. 16, the adhesive pad 35, the patch base 34, the base receiver 24, and the cartridge base 23 are provided so as to be stacked from the bottom to the top. The lower surface is provided with an opening 29. The opening 29 is a portion formed by forming a hole in a laminated structure of the adhesive pad 35, the patch base 34, and the base receiver 24. A surface of the cartridge septum 27 is exposed in the inner part thereof. In other words, the opening 29 is closed by the cartridge septum 27. The cartridge septum 27 communicates with the outflow portion 235 by the flow channel 236. FIG. 16 illustrates a state in which no liquid is present, and the storage portion 26 is not provided, and a reserve film 25 is in contact with the cartridge base 23. When the needle of the syringe is picked into the cartridge septum 27 and liquid is infused from the syringe into a flow channel 236, liquid is infused from the outflow portion 235, and the storage portion 26 is formed as illustrated in FIG. 2, so that the reserve film 25 comes into contact with the base receiver 24.

Since an outer surface of the cartridge septum 27 is arranged so as to face the biological body surface, the opening 29 is closed by the biological body surface when the liquid transport apparatus 1 is adhered to the biological body. Therefore, while the liquid transport apparatus 1 is adhered to the biological body, the cartridge septum 27 does not come into contact with the outside air, and hence there is no danger of deterioration or contamination caused by the outside air. If the cartridge septum 27 does not face the biological body surface and faces the side surface of the liquid transport apparatus 1 or upward, the cartridge septum 27 may be contaminated by the outside air.

In this manner, in the case where the outer surface of the cartridge septum 27 is arranged so as to oppose the biological body surface, if the outer surface of the cartridge septum 27 on the biological body side and an adhesive surface of the adhesive pad 35 (the mounting surface of the liquid transport apparatus 1 with respect to the biological body) are provided on the identical surface, and the outer surface of the cartridge septum 27 comes into direct contact with the biological body surface, the outer surface of the cartridge septum 27 may be contaminated by bacteria or the like on the biological body surface. In contrast, in this embodiment, the cartridge septum 27 is arranged at a position set back (upper side) from the biological body surface, and a gap is provided between the outer surface of the cartridge septum 27 on the biological body side and the adhesive surface of the adhesive pad 35 (the mounting surface of the liquid transport apparatus 1 with respect to the biological body). In other words, the cartridge septum 27 is away from the biological body surface. Accordingly, even though the liquid transport apparatus 1 is adhered to the biological body, the outer surface of the cartridge septum 27 does not come into contact with the biological body surface, so that the outer surface of the cartridge septum 27 is prevented from being contaminated by bacteria or the like.

In this embodiment, a C-chamfering is provided on the peripheral surface of the opening 29. Accordingly, the injection needle can be picked through the cartridge septum 27.

In this embodiment, the cartridge base 23 and the base receiver 24 are provided by being stacked in the direction of picking the needle into the cartridge septum 27, and the cartridge septum 27 is interposed between the cartridge base 23 and the base receiver 24. Accordingly, the cartridge septum 27 is arranged so as to be set back (upper side) from the biological body surface by a distance corresponding to the thickness of the base receiver 24. Accordingly, when the needle is picked into the cartridge septum 27, the cartridge base 23 supports the cartridge septum 27, and when the needle is pulled out from the cartridge septum 27, the base receiver 24 supports the cartridge septum 27, so that the stable fixation of the cartridge septum 27 is achieved.

### SECOND EMBODIMENT

The shape of the outflow portion 235 in a cross-sectional view taken along the line B-B is not limited to the shape as in the first embodiment. For example, the following shapes are also applicable.

FIG. 17A is a cross-sectional view taken along the line B-B of a second embodiment. FIG. 17B is a drawing illustrating a direction in which the liquid is flowed out from the outflow portion 235 in the second embodiment. FIG. 17A and FIG. 17B illustrate the outflow portion 235 of the second embodiment. In the second embodiment, part of the depression 235A of the outflow portion 235 has a flow channel having the same width as the opening 235B in the lateral direction, and the opening is enlarged in the lateral direction in the vicinity of an area in the depression 235A connected to the storage portion 26.

Even with the shape described above, the liquid flowed into the depression 235A is dispersed in a portion enlarged in the lateral direction. Accordingly, the liquid flows in so as to spread to the left and the right direction also in the storage portion 26. The liquid flowed therein spreads over the entire curved shaped portion 23A in the storage portion 26. When the liquid is further flowed inward, the entire liquid spread over the entire part of the curved shaped portion 23A pushes the reserve film 261 upward (downward in FIG. 11). With this configuration as well, the force of the liquid flowing in is increased so that liquid can be flowed in efficiently against the inner pressure of the storage portion 26.

### THIRD EMBODIMENT

In the first embodiment described above, the depression 235A is provided in the outflow portion 235, and the liquid is dispersed in the depression 235A. However, the liquid can be flowed in efficiently by increasing the flow rate of the liquid even without providing the depression 235A.

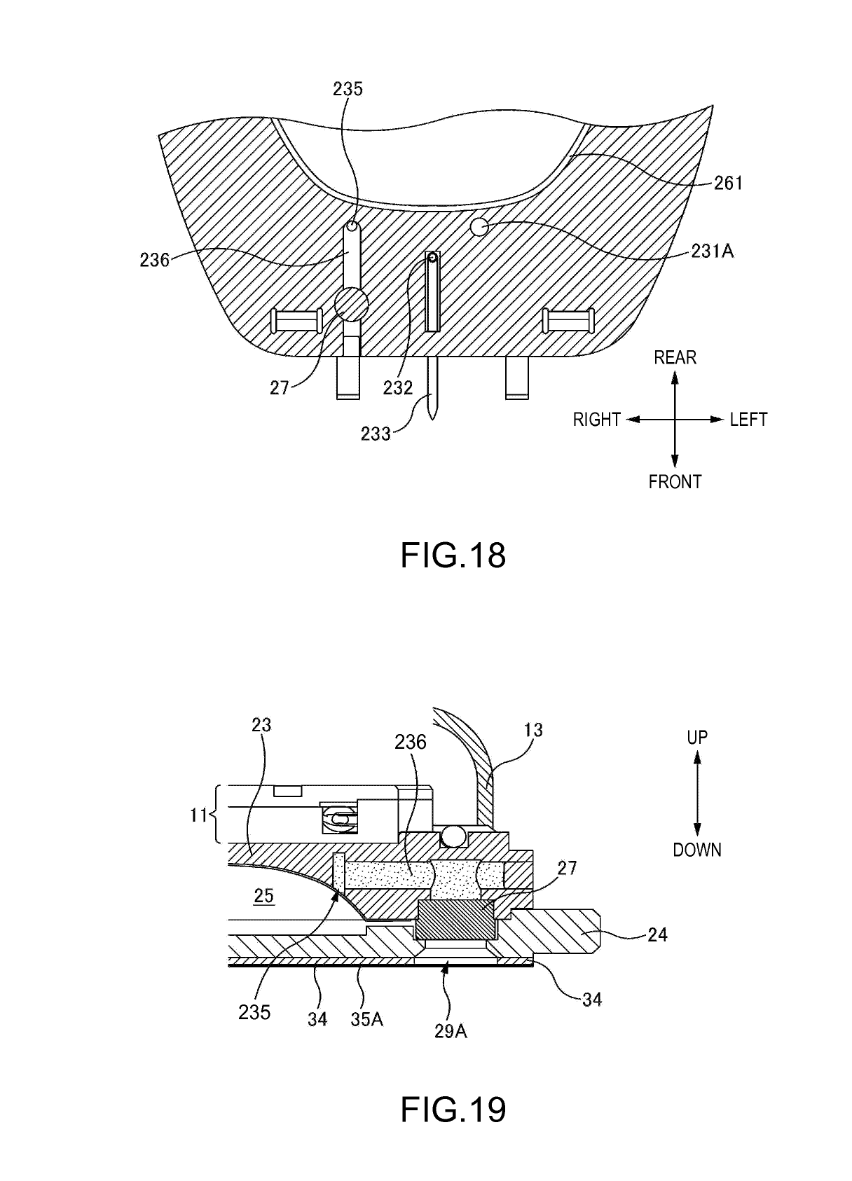

FIG. 18 is a cross-sectional view of FIG. 11 of a third embodiment taken along the line A-A. FIG. 18 is different in that the depression 235A having the slit-shaped portion 235C is not provided in the outflow portion 235, and the cylindrical outflow portion 235 communicating with the flow channel 236 is directly connected to the storage portion 26. However, the cross-sectional area of the outflow portion 235 is smaller than the flow channel 236.

With this configuration as well, since the flow rate on the opening 235B side can be set to be higher than that on the inflow portion 237 side, the liquid can be flowed into the storage portion 26 efficiently against the internal pressure therein.

### FOURTH EMBODIMENT

FIG. 19 is a cross-sectional view of the liquid transport apparatus 1A including the cartridge septum 27 of a fourth embodiment. In the liquid transport apparatus 1A, the opening 29A is a portion formed by forming a hole in a laminated structure of the base receiver 24 and the patch base 34, and the peripheral surface of the opening 29A is provided with the C-chamfering. The patch base 34 having a hole is covered with the adhesive pad 35A having no hole. A double-sided adhesive tape is used as the adhesive pad 35A.

In other words, in the first embodiment described above, the adhesive pad 35 is provided with the hole, and the cartridge septum 27 is exposed. In contrast, in the fourth embodiment, the adhesive pad 35A has no hole, and the opening 29A is covered with the adhesive pad 35A.

A procedure of usage of the liquid transport apparatus 1A will be described below.

(1) First of all, in a state in which the adhesive pad 35A is not present (in a state in which the cartridge septum 27 is exposed), the needle of the syringe is picked into the cartridge septum 27, and liquid is infused into the storage portion 26.

(2) After the liquid has infused into the storage portion 26, one of the surfaces of the adhesive pad 35A is adhered to the patch base 34.

(3) Subsequently, the other surface of the adhesive pad 35A is adhered to the biological body.

Accordingly, since the liquid transport apparatus 1A is adhered to the biological body in a state in which the cartridge septum 27 is covered with the adhesive pad 35A, isolation from the outside is achieved, and hence the outer surface of the cartridge septum 27 can hardly be contaminated by bacteria or the like. Since the outer surface of the cartridge septum 27 and the upper surface of the adhesive pad 35A do not come into contact with each other, the adhesive pad 35A is not adhered to the outer surface of the cartridge septum 27.

### FIFTH EMBODIMENT

In the embodiment described above, the liquid is pumped by a pump of the type compressing the tube 21 by the plurality of fingers 22 in sequence. However, the type of the pump is not limited thereto.

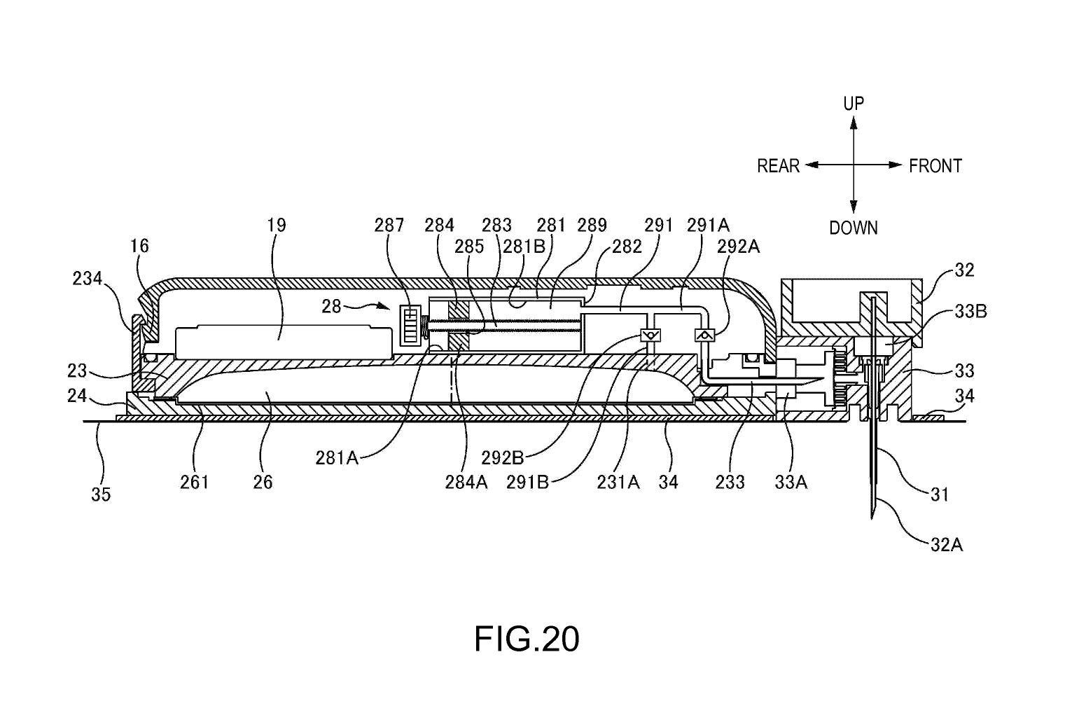

FIG. 20 is a cross-sectional view of the liquid transport apparatus 1 of a fifth embodiment. In the fifth embodiment, the type of the pump is differentiated from the embodiments described above. However, other configurations are the same as the embodiments described above. Therefore, a syringe pump 28 of the fifth embodiment will be described with reference to FIG. 20.

The syringe pump 28 includes a cylindrical portion 281, an end wall 282, a parent screw 283, a plunger 284, an insert 285, and a gear 287. As described later, the syringe pump 28 includes a small gear (not illustrated) configured to engage the gear 287 and a motor (not illustrated) for rotating the small gear.

A side wall 281B in the interior of the cylindrical portion 281 has an inner wall having a substantially cylindrical shape extending in the coaxial direction with the parent screw 283. The substantially cylindrical plunger 284 is inserted so as to come into tight contact with the side wall 281B. The insert 285 is secured to the inside of the plunger 284. The insert 285 is provided with an inner screw which is engageable with the parent screw 283. Then, a liquid chamber 289 is formed between the plunger 284 and the side wall 281B and the end wall 282.

The plunger 284 is provided with a slide projection 284A extending in the coaxial direction with the parent screw 283. In contrast, the side wall 281B is provided with a slide depression 281A configured to engage the slide projection 283A. Accordingly, the rotation of the plunger 284 is limited, and in contrast, the movement of the plunger 284 in the coaxial direction with the parent screw 283 is allowed.

The parent screw 283 extends in the cylindrical portion 281 from the end wall 282 to an open port side of the cylindrical portion 281. The gear 287 configured to be rotatable coaxially is fixed to the end portion of the parent screw 283 on the open port side. The small gear, which is not illustrated, engages the gear 287. To this small gear, a motor, which is not illustrated, is connected. In this configuration, the rotation of the motor is controlled so that the rotation of the parent screw 283 can be controlled.

A tube 291 is connected to the end wall 282. The tube 291 is branched in mid-course, and a tube 291A, which is one of the branches, is connected to the connecting needle 233. A tube 291B, which is the other one of the branches, is connected to the supply channel 231A communicating with the storage portion 26.

The tube 291A is provided with a one-way valve 292A configured to allow the movement of the liquid from the syringe pump 28 side to the connecting needle 233 side. The tube 291B is provided with a one-way valve 292B configured to allow the movement of the liquid from the storage portion 26 side toward the syringe pump 28.

In this configuration, the liquid can be flowed into the liquid chamber 289 from the storage portion 26 side by moving the plunger 284 in the direction in which the liquid chamber 289 is enlarged. In contrast, the liquid can be flowed from the liquid chamber 289 to the connecting needle side by moving the plunger in the direction in which the liquid chamber 289 is reduced. Accordingly, the number of rotation of the parent screw 283 is controlled, and hence the liquid can be infused into the object of infusion.

### OTHER EMBODIMENTS

The embodiment described above is for facilitating the understanding of the invention, and is not for interpreting the invention in a limited range. It is needless to say that the invention may be modified or improved without departing from the scope of the invention and equivalents are included in the invention.

The entire disclosure of Japanese Patent Application Nos. 2013-262143, filed Dec. 19, 2013, 2013-262144, filed Dec. 19, 2013 and 2013-262145, filed Dec. 19, 2013 are expressly incorporated by reference herein.

What is claimed is:

1. A liquid transport apparatus comprising:
a storage portion configured to store liquid; and
an outflow portion arranged on an inner wall of the storage portion,
wherein the outflow portion includes a depression arranged on the inner wall and an opening arranged on a bottom portion of the depression.

2. The liquid transport apparatus according to claim 1,
wherein the inner wall of the storage portion has an inclined surface shape,
the depression includes a slit-shaped portion formed to be an opening at the same height on the inclined surface.

3. The liquid transport apparatus according to claim 1,
wherein the liquid is supplied from the opening to the depression and flows into the storage portion.

4. The liquid transporting apparatus according to claim 1,
wherein the opening is arranged at a center of the depression.

5. The liquid transport apparatus according to claim 1,
wherein the storage portion includes a curved shaped portion and a film arranged so as to extend along the curved shaped portion, and the liquid is stored between the curved shaped portion and the film.

6. The liquid transport apparatus according to claim 1, further comprising:
a pump configured to transport the liquid to an object of infusion,
wherein the storage portion is provided with a supply channel configured to supply the liquid to the pump.

7. The liquid transport apparatus according to claim 1, further comprising:
a base on which the storage portion is arranged; and
a flow channel connected to the openings,
wherein the flow channel is arranged on the base.

8. The liquid transport apparatus according to claim 7, further comprising:
a lid member arranged in the flow channel.

9. The liquid transport apparatus according to claim 1, further comprising:
an inflow portion configured to allow the liquid to flow inward; and
a flow channel configured to connect the inflow portion and the outflow portion,
wherein a flow rate of the liquid is set to be higher on the outflow portion side than the inflow portion side.

10. The liquid transport apparatus according to claim 9,
wherein a lid member is arranged in the flow channel.

11. The liquid transport apparatus according to claim 10,
wherein the lid member is a septum.

12. The liquid transport apparatus according to claim 9,
wherein the cross-sectional area on the outflow portion side is set to be smaller than the cross-sectional area on the inflow portion side.

13. The liquid transport apparatus according to claim 9,
wherein the storage portion includes a curved shaped portion and a film arranged so as to extend along the curved shaped portion, and the liquid is stored between the curved shaped portion and the film.

14. The liquid transport apparatus according to claim 9,
wherein the inner wall of the storage portion has an inclined surface shape, and
the depression includes a slit-shaped portion formed to be an opening at the same height on the inclined surface.

15. The liquid transport apparatus according to claim 9, further comprising:
a base on which the storage portion is arranged,
wherein the flow channel is arranged on the base.

16. The liquid transport apparatus according to claim 1, further comprising:
a lid member configured to close an opening which opens toward the biological body and configured to allow a needle for infusing the liquid into the storage portion to pick therethrough,
wherein a gap is provided between an outer surface of the lid member on the biological body side and a mounting surface of the liquid transport apparatus with respect to the biological body.

17. The liquid transport apparatus according to claim 16,
wherein the opening is covered with an adhesive sheet configured to attach the liquid transport apparatus to the biological body.
